(19) United States
(12) Patent Application Publication
(10) Pub. No.: US 2024/0200005 A1
(43) Pub. Date: Jun. 20, 2024

(54) METHOD FOR DEVELOPING AND/OR REPROGRAMMING PLANT CELLULAR OBJECTS

(71) Applicant: SCREENSYS GMBH, Freiburg i.BR (DE)

(72) Inventors: Jialan CAO, Ilmenau (DE); Alexander GROSS, Ilmenau (DE); Johann Michael KÖHLER, Golmsdorf (DE); Patrick SCHAUB, Freiburg i. Br. (DE); Klaus PALME, Freiburg i. Br. (DE); Oleksandr DOVZHENKO, Freiburg i. Br. (DE)

(21) Appl. No.: 18/555,347

(22) PCT Filed: Apr. 14, 2022
(86) PCT No.: PCT/EP2022/060012

§ 371 Date: Oct. 13, 2023

(30) Foreign Application Priority Data
Apr. 15, 2021 (EP) 21168659.7
Mar. 7, 2022 (EP) 22160607.2

(51) Int. Cl. C12M 3/06; C12M 1/00; C12M 1/34; C12N 5/04; G01N 15/1434; G01N 15/149; G01N 33/00

(52) U.S. Cl. CPC C12M 23/16; C12M 41/46; C12M 47/04; C12N 5/04; G01N 15/1434; G01N 15/149; G01N 33/0098; C12N 2523/00; C12N 2529/10

(57) ABSTRACT

The present invention relates to a method for developing and/or reprogramming plant cellular objects comprising the steps: providing a reservoir containing a medium with plant cellular objects: providing a first set of compartments of sample fluid embedded in carrier fluid in a microfluidic conduit, wherein the carrier fluid is immiscible with the medium, wherein the first set's compartments of sample fluid each comprise medium and at least one plant cellular object: providing one or more first state triggers to the plant cellular objects in the microfluidic conduit for inducing a first state in the plant cellular objects of the first set of compartments: incubating the plant cellular objects of the first set of compartments in the microfluidic conduit for a time span sufficient for the plant cellular objects to transfer to the first state: selecting one or more first selection parameters indicative of the first state: identifying, within the first set of compartments in the microfluidic conduit, compartments according to the one or more first selection parameters and optionally assigning the compartments with respective state identifiers.

The present invention relates to a method for developing and/or reprogramming plant cellular objects.

In view of climate change and an exploding human population, development of plants with specific properties is a focus in agriculture. To date, plant breeding techniques are not only time consuming but also require many working hours and are often based on small scale experiments.

There remains a need for high throughput screening and/or high throughput production of plant cellular objects with specific properties amenable to high degree of process automation.

The present invention relates to a method for developing and/or reprogramming plant cellular objects comprising the steps:
a) providing a reservoir, e.g. a microreservoir, containing a medium with plant cellular objects;
b) providing a first set of compartments of sample fluid embedded in carrier fluid in a microfluidic conduit, wherein the carrier fluid is immiscible with the medium, wherein the first set's compartments of sample fluid each comprise medium and at least one plant cellular object;
c) providing one or more first state triggers to the plant cellular objects in the microfluidic conduit for inducing a first state in the plant cellular objects of the first set of compartments;
d) incubating the plant cellular objects of the first set of compartments in the microfluidic conduit for a time span sufficient for the plant cellular objects to transfer to the first state;
e) selecting one or more first selection parameters indicative of the first state;
f) identifying, within the first set of compartments in the microfluidic conduit, compartments according to the one or more first selection parameters and optionally assigning the compartments with respective state identifiers.

The method is suitable for high throughput screening and/or high throughput production of plant cellular objects with specific properties at high degree of process automation.

Herein, the term “reprogramming” is used to refer to the transfer a plant cellular object from its current state to another state. “State” may be used for differentiated plant cells, such as pollen precursor cell microspores, or protoplasts from various plant tissues in particular, or dedifferentiated cells or callus cells. “State” may refer to a developmental stage and/or a developmental sub-stage of the plant cellular object, as distinguishable by any suitable indicator, e.g. a change in morphology and/or biochemical indicators. Examples are dedifferentiation or gaining competence to acquire a new cell fate or gaining a new cell fate, cell division and subsequent further cell divisions, growth by cell division and expansion, enable embryogenesis (such as formation of globular, heart or torpedo or embryo proper structures), or organogenesis (such as formation and functional use of meristems, form functional root or shoot structures). The term “plant cellular object” refers to any object originating from a plant, particularly to an individual cell, a cell cluster, a part of a plant cell, e.g. protoplast, microspore, and a multicellular structure such as an embryo or an organoid. The size in terms of the largest dimension (e.g., diameter) of the plant cellular objects is preferably 1 mm or smaller.

The medium may be selected according to the plant cellular objects. The medium may comprise nutrition component(s) and/or have pH buffering properties. The medium is preferably water based. The reservoir containing the medium may have any suitable size and may be adapted to the number and volume of compartments that are intended to be provided.

Phase A is “immiscible” with phase B means, that phases A and B stay separated for a time sufficiently long to carry out the experiments.

Preferably, the carrier fluid is immiscible with the sample fluid.

The microfluidic conduit may have any suitable size. Preferably, it has an inner width or inner diameter of 5 mm or less, more preferably 3 mm or less, more preferably 2 mm or less, more preferably 1.5 mm or less, most preferably 0.5 mm. Preferably, the microfluidic conduit has an inner diameter of at least 5 μm, more preferably of at least 20 μm. The inner diameter may be selected according to the size of the plant cellular object and microfluidic considerations in order to ensure that compartments of sample fluid embedded in carrier fluid and comprising the plant cellular objects are stable. In microfluidic conduits with non-circular cross-section, the term “width” preferably refers to the largest dimension of the lumen in a cross section perpendicular to the longitudinal extension of the conduit.

A set of compartments may comprise any suitable number of compartments, e.g. least 1, at least 2, at least 5, at least 10, at least 100, at least 1000 or at least 10000 compartments of sample fluid.

The term “state trigger”, or short “trigger”, refers to anything that causes or potentially causes a reaction of the plant cellular objects, in particular a transition of the plant cellular objects from one state to another. For example, a chemical and/or biological substance, light, temperature, pH, and the like may be a state trigger.

“Selection parameter” refers to anything that may serve as an indicator of the induced specific state of the plant cellular object. For example, morphology such as size and or shape, “alive” vs. “dead”, a state-dependent internal, including endogenously encoded, reporter or external reporter such as a color or a live staining dye, for example a fluorescence marker such as FDA, FM4-64 or others, a pigment particle or plasmonic nanoparticles may be a selection parameter. The selection parameter may refer to properties of the plant cellular object, the compartment and/or additional markers in the compartments. For example, one or more markers in the compartment may change color to indicate a change in pH. Such change in pH may indicate a change in metabolism of the plant cellular object, which may be indicative of a change in state.

If the selection parameter(s) are fulfilled, the compartments are identified as such and may be assigned with respective state identifiers. A state identifier may be a virtual identifier, e.g. a number, tag, or the like, e.g. in an electronic file or random access memory. A state identifier may, alternatively or additionally, be a chemical and/or biological substance/object, e.g. one or more beads, fluorescent dyes, and/or the like.

Preferably, the identification takes place while the first set of compartments is in the microfluidic conduit.

The method may further comprise the step:
g) sorting the first set's compartments according to their state as identified in step f), e.g. separating the compartments identified in step f) as comprising plant cellular objects that have, according to the one or more first selection parameters, transferred to the first state from the compartments that were not identified in step f) as comprising plant cellular objects that have transferred to the first state, preferably sorting the first set's compartments according to their state identifiers.

Herein, an alphabetical numbering is used for the method steps. This has the main purpose of identifying the method steps so as to be able to refer to the steps without repeating the wording of the respective step word by word. It is emphasized that the alphabetical order may be a suitable order of performing the method steps, or even a preferred order from the viewpoint of a particular compartment. However, any other suitable order is contemplated. Particularly, method steps may be carried out one after the other and/or concurrently. For example, and especially when dealing with a large number of compartments, step b), i.e. the formation of compartments, may still be ongoing, while compartments that were produced earlier are already subject to one or more of method steps c)-f). Such a concurrent implementation of method steps may be preferred, particularly for large numbers of compartments and/or sets of compartments, as will be explained below. This also applies to method steps that are not labeled with an alphabetical number and/or sub-steps of method steps.

The method may further comprise:
h) providing a second set of compartments of sample fluid embedded in carrier fluid in the microfluidic conduit, wherein the second set's compartments of sample fluid each comprise medium and at least one plant cellular object;
i) providing one or more second state triggers to the second set's plant cellular objects in the microfluidic conduit for inducing a second state in the plant cellular objects of the second set of compartments;
j) incubating the plant cellular objects of the second set of compartments in the microfluidic conduit for a time span sufficient for the plant cellular objects to transfer to the second state;
k) selecting one or more second selection parameters indicative of the second state;
l) identifying, within the second set of compartments in the microfluidic conduit, compartments with plant cellular objects that have transferred to the second state and optionally assigning the compartments with respective state identifiers.

The method may further comprise the step:
m) sorting the second set's compartments according to their state as identified in step l), e.g. separating the compartments identified in step l) as comprising plant cellular objects that have, according to the one or more second selection parameters, transferred to the second state from the compartments that were not identified in step
l) as comprising plant cellular objects that have transferred to the second state, preferably sorting the second set's compartments according to their state identifiers.

A set of compartments may include one or more compartments without any plant cellular object, which may also be referred to as so called “empty compartments” herein.

It is also contemplated that there may be one or more empty compartments between two subsequent sets of compartments with cellular objects or microobjects. This may be helpful, for example, for later identifying where each set of compartments starts and ends.

Alternatively, some of the compartments may contain another detectable microobject or probe, e.g. a fluorescent bead, which may help to organize an order of compartments in a logical manner, such as the first 10 or 100 or else compartments being separated from the next 10 or 100 or else compartments and so on.

So far, the invention has been described by referring to a first and a second set of compartments. The method is also suitable for a larger number of sets of compartments. As such, the present invention also relates to a method for developing and/or reprogramming plant cellular objects comprising the steps:
a) providing a reservoir containing a medium with plant cellular objects;
b) providing several sets of compartments of sample fluid embedded in carrier fluid in a microfluidic conduit, wherein the carrier fluid is immiscible with the medium, wherein the compartments of sample fluid comprise at least one plant cellular object each;
c) providing respective state triggers to the plant cellular objects in the microfluidic conduit for inducing respective states in the plant cellular objects;
d) incubating the plant cellular objects in the microfluidic conduit for a time span sufficient for the plant cellular objects to transfer to the respective state;
e) for each state, selecting one or more selection parameters indicative of the state;
f) identifying, within the several sets of compartments, compartments with plant cellular objects that have transferred to the respective state(s) and optionally assigning the compartments with a state identifier.

As previously mentioned, the method may further comprise the step:
g) sorting the compartments according to their states as identified in step f), e.g. separating the compartments identified in step f) as comprising plant cellular objects that have transferred to the respective state from the compartments that were not identified in step f) as comprising plant cellular objects that have transferred to the respective state, preferably sorting the compartments according to their state identifiers.

Preferably, the size and shape of the microfluidic conduit and the size/volume and shape of the compartments are selected such that the compartments, e.g. within a set of compartments, are provided in a row in a defined order in the microfluidic conduit. Particularly preferably the compartment's and conduit's size are such that in the microfluidic conduit each two consecutive compartments are separated from each other by carrier fluid. Between the two consecutive compartments, the carrier fluid fills the entire cross section of the tube. As such, the carrier fluid may also be considered to form plugs of carrier fluid in the conduit that completely separate one compartment from the next, and/or vice versa, the compartments may be considered to form plugs separating the carrier fluid. In other words, each compartment fully occupies a cross-section of the microfluidic conduit and/or each plug of carrier fluid fully occupies a cross-section of the microfluidic conduit.

Preferably the carrier fluid is immiscible with the sample fluid(s) of the compartments.

As already mentioned, the plant cellular objects may be selected from: protoplasts, microspores, plant pollen cells, seeds, fragments originating from whole plants, such as fragments from root, stem, petals, leaves, etc., suspension or callus cells and small cell clusters including multicellular structures such as embryos and organoids.

The method may comprise: assigning a compartment identifier and/or a set identifier to the compartments. As the state identifiers, the compartment identifier and/or the set identifier may be a virtual identifier, e.g. a number, tag, or the like, e.g. in an electronic file or random access memory. The compartment identifier and/or the set identifier may be a chemical and/or biological substance/object, e.g. one or more beads, fluorescent dyes, and/or the like. Any combination of identifier types for state, compartment and set identifiers is contemplated. The set identifier is indicative of a specific set or group of sets. It may be applied to every compartment or only a subgroup, e.g. the first one, of the compartments of the respective set. The compartment identifier is indicative of a specific compartment within one set and/or within all the compartments in the microfluidic conduit. For example, a microfluidic conduit may house 3 sets of compartments, with 10 compartments each, i.e. a total of 30 compartments. Then, in order to identify the compartments, each compartment may have a corresponding number, i.e. 1-30, in a table. The first, the eleventh and the 21^{st} compartment may be labeled with one, two and three fluorescent beads as set identifiers, respectively. Alternatively, all compartments of the first set, i.e. compartment no. 1 to compartment no. 10 may be labeled with one fluorescent bead, all compartments of the second set, i.e. compartment no. 11 to compartment no. 20 may be labeled with two fluorescent beads, and all compartments of the third set, i.e. compartment no. 21 to compartment no. 30 may be labeled with three fluorescent beads. Additionally, or alternatively to using different numbers, different colors of beads may be used. An identifier serving as a set identifier may also be a compartment identifier.

After identification, the compartments may be treated, e.g. sorted and/or further triggered, accordingly. For example, the method may further comprise the step:
transferring the sample fluid compartments to a subsequent procedural step in accordance with:
   i. their compartment identifier;
   ii. their set identifier; and/or
   iii. their state identifier;
and/or
discarding the compartments with a state identifier that indicates that the compartment was not identified as comprising plant cellular objects that have transferred to the respective state.

The sample fluid compartments and/or sets of sample fluid compartments may be treated individually, preferably according to their compartment identifier and/or set identifier and/or state identifier.

Compartments that have been identified to be no longer of use, may be discarded. Compartments that have transferred to the respective state may be kept for further processing. For example, each suitable compartment may receive a further, individual, trigger according to its identifier(s). Alternatively, or additionally, suitable compartments may be incubated to reach a certain developmental state. Alternatively, or additionally, data on suitable compartments may be collected and analyzed in order to identify suitable triggers during screening.

Protoplasts, microspores, plant pollen cells, seeds, fragments originating from whole plants, such as fragments from root, stem, petals, leaves, etc., suspension or callus cells and small cell clusters including multicellular structures such as embryos and organoids may be used as plant cellular objects and compartmentalized. One or several different chemical substances may be added to sets of compartments, optionally at different concentrations. Additionally or alternatively, other triggers such as specific temperature or light regimes, e.g. heat shock, cold shock and/or light regime and/or light spectrum, may be applied. This may be used for screening purposes and/or, if the impact of a trigger is already known, for production purposes.

The present invention also relates to/includes screening for optimized trigger(s) for inducing double haploid plant pollen cells, i.e. type of trigger, particularly specific substances, trigger concentration, incubation periods and the like.

The present invention also relates to/includes producing haploid, aneuploid, doubled haploid or polyploid cell mass, embryo structures, organoids or plants from microspores. Particularly, microspores may be used as plant cellular objects, and heat shock, small chemicals, e.g. epigenetic drugs, phytohormones and/or anti-mitotic drugs may be provided as state trigger(s) to the microspores in the compartments. The plant cellular objects, e.g. microspores, of compartments in the microfluidic conduit may be incubated for a time span sufficient for the microspores to transfer to the other state such as cell mass, embryo structures or organoids. The size of the plant cellular objects, e.g. of the microspores, or of what the microspores have developed into, may be selected as a selection parameter indicative of the embryo state. Within the microfluidic conduit, compartments may be identified according to the selection parameter “size”, i.e. those compartments with microspores that have reached the embryo state. Optionally, the compartments may be assigned with respective state identifiers.

Production purposes can be, but are not limited to, generation of new haploid, aneuploid, doubled haploid or polyploid cell mass, embryo structures, organoids or plants or at least some steps of the generation of haploid, aneuploid, doubled haploid or polyploid cell mass, embryo structures, organoids or plants.

One way of generating new haploid, aneuploid, doubled haploid or polyploid cell mass, embryo structures, organoids or plants in the lab is the reprogramming of microspores. As a stage in pollen development, microspores of flowering plant species can be reprogrammed in vitro by stress treatment, such as heat shock, starvation or chemical treatment and androgenesis can be induced. This means that the microspores start to develop into embryos and later on into full plant, without any kind of fertilization. Thus, deriving only from male germ cell precursors, obtained embryos are haploid. However, either randomly or induced, e.g. by treatment with certain substances such as colchicine, aneuploid, or homozygous doubled haploid or polyploid cell mass, embryo structures, organoids or plants and ultimately fertile plants can be generated. This is highly advantageous for plant breeding, because of preservation of the genotype in daughter generations due to the lack of genetic recombination, which happens during sexual reproduction.

The one or more trigger(s) may be provided by changing the composition of the sample fluid in the compartments as compared to the medium upon formation of the sample fluid compartments, i.e. the composition may be changed while the respective compartment is formed. Additionally, or alternatively, the trigger(s) may include changing the composition of the sample fluid in the compartments after formation of the sample fluid compartments while the compartments are in the microfluidic channel. In either case, changing the composition may be done by adding a suitable fluid to the compartments, optionally including a chemical trigger. By adding further fluid, e.g. sample fluid, the concentration of substances already present in the compartments may be reduced.

Particularly, changing the composition may include adding a manipulation fluid comprising a trigger substance. The manipulation fluid may be added to the compartments, while the compartments are in the microfluidic channel. Any suitable microfluidic device may be used.

Different sets of sample compartments may be provided with different trigger substances. Additionally, or alternatively, different compartments of sample fluid within the first and/or second set may be provided with the same trigger substance at different concentrations.

The compartments may comprise 1000 or less, 200 or less, preferably 50 or less, more preferably 10 or less plant cellular objects per compartment. The number of plant cellular objects per compartment may be selected according to the volume of the compartments, and vice versa. Both values may be selected in accordance with the identification technique for identifying the compartments according to the one or more selection parameters. The values may be selected such that an optimal analysis is achieved. All or only a certain percentage of plant cellular objects may be analyzed as desired. For example, analyzing statistical samples, i.e. only some and not all of the plant cellular objects of a compartment, may be sufficient to decide on the state identifier of a compartment and/or on the fate of the compartment. As another example, the chosen identification technique may not be able to analyze all plant cellular objects of a compartment. This may, for example, occur with optical detection techniques, where some plant cellular objects may be covered by other plant cellular objects of the compartment.

The compartments may each have a volume of:
at least 10-30 nL, or 30-60, or at least 60-100 nL;
at least, 10 nL, at least 30 nL, at least 60 nL, or at least 100 nL; and/or
10 μl or less, preferably 5 μl or less.

Preferably, the compartments may each have a number density of 50 plant cellular objects per 100 nL compartment volume. However, the number density may also depend on the size of the plant cellular objects: the smaller the size, the more plant cellular objects may, at least theoretically, be in a specific volume.

As generally known in the art, the state trigger depends on the type of plant cellular object and/or on the species it is derived from. The state trigger may comprise at least one substance selected from the group comprising: culture medium salts or organic components, small chemical molecules, plant growth regulators or macromolecules, temperature and light regimes, —or combinations of different triggers.

The microfluidic conduit may have any suitable shape. For example, the microfluidic conduit may be a tube, wherein the microfluidic conduit/tube is preferably at least 10 cm long, more preferably at least 50 cm long, more preferably at least 100 cm long, even more preferably at least 500 cm long. The tube may be even longer. The term “tube” refers to a flexible, elongated conduit, which may also be referred to as a hose. The tube may be wound onto a spool.

The microfluidic conduit, in particular a material from which the tube is formed, may be at least partially or entirely transparent. The term “transparent” is understood as meaning sufficiently transparent for the respective observation technique. For example, if the observation technique is microscopy with visible light, the tube is considered transparent, if images with light of visible wavelengths may be taken and the image quality is sufficient for further analysis.

Alternatively or additionally, the tube material may have a certain gas permeability for CO₂ and/or for O₂ that is sufficient for supplying the plant cellular objects with the necessary amount of CO₂ and/or for O₂.

The tube material may be low wetting with water. Preferably, “low wetting” refers to a material forming, in ambient atmosphere, a contact angle with water of more than 90°, preferably more than 120°. This is also meant with “poorly wettable” and “poor wettability”.

The tube material may be polymeric, preferably wherein the tube material comprises or consists of fluorethylen-propylen or polytetrafluorethylene (PTFE).

The plant cellular objects may be haploid microspore stem cells, for example haploid microspore stem cells of Brassica napus. The potentially induced state may be a plant embryo. The term “plant embryo” refers to an early stage of development of a multicellular organism like a plant. In general, in organisms that reproduce sexually, embryonic development is the part of the life cycle that begins just after fertilization and continues through the formation of body structures, such as tissues and organs. Embryos can also be formed from single cells like microspores or even protoplasts by inducing embryonic competence, cell division, cell differentiation and subsequent organ formation (i.e. roots and shoots).

The method according to the invention is suitable for small scale application as well as for large scale applications. Step b)/h) may include: providing at least 100, preferably at least 1000, more preferably at least 10000 compartments per tube. The microfluidic conduit, particularly the tube, itself may be used for incubation, i.e. the compartments may be located in the microfluidic conduit/tube.

A combination of two or more of the following parameters may be selected in accordance with one another: length of the microfluidic conduit, particularly the tube; inner diameter of the microfluidic conduit, particularly the tube; the volume of the compartments; the number of compartments. For example, a microfluidic conduit with a length of 100 cm and an inner diameter of 0.5 mm may house compartments with a volume of 0.1 to 5 μL, preferably between 0.1 μL and 1.0 μL. In this case, the microfluidic conduit may house preferably 100, more preferably 500, up to 1000 microfluidic compartments. In case of a tube of 0.2 mm inner diameter, a preferable compartment volume is about 50 nl and 100 cm of tube contains about 2000 compartments. With compartments of 600 nl volume each, 100 cm of the tube may house 100 compartments. With compartments of 400 nl each, a tube of 100 cm length may house 200 compartments. In case of a chip based storage device with compartment (droplet) volumes of less than 100 nl may be used, and/or droplet numbers of 1000 up to 10.000 droplets may be used.

Any suitable microfluidic system may be used for the method steps. For example, step b)/h) may include:
i. providing a sample of dispersed plant cellular objects in the medium;
ii. providing the microfluidic conduit with an inlet, an outlet and an opening located between the inlet and the outlet;
iii. providing the carrier fluid via the inlet to the microfluidic conduit at an input volumetric flow rate (Qᵢₙ) and removing fluid from the microfluidic conduit via the outlet at an output volumetric flow rate (Qₒᵤₜ);
iv. positioning the opening in the medium;
v. setting the input volumetric flow rate (Qᵢₙ) to be smaller than the output volumetric flow rate (Qₒᵤₜ), the difference resulting in a takeup volumetric flow rate (Qₜₐₖₑᵤₚ) of medium through the opening that results in the medium being embedded in the carrier fluid as the sample fluid compartments.

Particularly, if the microfluidic conduit is a tube, this tube may comprise the opening mentioned in step ii), while a first end of the tube serves as the inlet and a second end of the tube serves as the outlet. Such system is described, e.g., in WO 2010/142471 A1.

The plant cellular objects and/or compartments may be selected and/or manipulated as required. For example, step b)/h) may further include:
vi. selecting a target plant cellular object among the plant cellular objects and changing a position of the opening relative to the target plant cellular object from a starting relative position to a target position to bring the opening proximate to the target plant cellular object;
vii. drawing the target plant cellular object into the microfluidic conduit together with a respective compartment of sample fluid when embedding the compartment of sample fluid in the carrier fluid according to step v);
viii. repeating steps i)-vii) until a desired number of compartments has been created.

The microfluidic conduit may comprise a manipulation inlet arranged at the opening or between the opening and the outlet, and the method may further comprise: delivering a manipulation fluid via the manipulation inlet into a respective compartment in the microfluidic conduit at a manipulation volumetric flow rate Qₘₐₙᵢₚ, wherein the manipulation fluid optionally comprises a trigger substance.

Alternatively, or additionally, step b)/h) may include:
i. providing a microfluidic manifold comprising a microchannel, the microchannel being in fluid communication with the microfluidic conduit, a carrier fluid supply, the reservoir, and optionally a further fluid supply;
ii. adjusting a flow rate from the carrier fluid supply to the manifold, a flow rate from the reservoir to the manifold, and optionally a flow rate from the further fluid supply to the manifold such that compartments of sample fluid embedded in carrier fluid are created in the microchannel of the manifold, wherein the compartments of sample fluid each comprise medium and at least one plant cellular object;
iii. transferring the compartments of sample fluid from the microchannel of the manifold to the microfluidic conduit.

In other words, a stream of sample fluid provided via a first microfluidic channel may be combined with a stream of medium comprising plant cellular objects provided via a second microfluidic channel. The combination may occur at a manifold. Compartments may be created by switching one or more valves that regulate the flow of carrier fluid and/or the flow from the reservoir. Alternatively, or additionally, by selecting adequate volumetric flow rates, matched with the geometries of the microfluidic channels and the manifold, a stream of compartments embedded in carrier fluid may be created. More than two fluid streams may be combined in a manifold.

The microfluidic conduit may be in fluid communication with and/or comprise a manifold, a valve, a flow focusing device, a nozzle and the like.

During creation of the compartments in the microfluidic conduit/tube, the microfluidic conduit/tube may be in an open configuration, i.e. it has two ends, which are separated from each other. After creation of the compartments, the microfluidic conduit/tube may be closed, i.e. the two ends may be connected, e.g. via an adapter or connector, to form a closed loop. The adapter or connector may comprise an inlet and or an outlet. Thus, in the closed loop configuration it is still possible to add or remove fluid. The closed loop configuration is particularly suitable for incubation periods.

It may be advantageous to provide accessories for opening the microfluidic conduit at one or more desired points. For example, if a compartment is contaminated with an undesired substance or unintended microorganisms, such as bacteria, the contaminated compartment may be removed through such an opening, while the remainder of the compartments may be kept in the microfluidic conduit. Additionally, or alternatively, the portion of the microfluidic conduit, that was housing the contaminated compartment may be completely removed. In order to be able to remove a compartment and/or a portion of the conduit, the remainder must be fluidly disconnected from the portion with the contaminated compartment, e.g. with clamps. Then the contaminated portion of the microfluidic conduit and/or the contaminated compartment may be removed. Afterwards, the hole may be sealed again and/or the clamped ends, which are the result of removing a portion of the microfluidic conduit, may be reconnected, e.g. with a respective connector.

The incubation step d) may include:
maintaining the temperature of the compartments above standard incubation temperature, preferably for at least 2 hours, at least 5 hours, at least 8 hours, at least 12 hours, at least 24 hours, at least 2 days, at least 3 days, at least 5 days or at least 7 days; and/or
reverse light conditions as compared to standard light conditions;
one or more further conditions, e.g. CO₂ concentration, which deviate from respective standard conditions.

Identification step e) may include: Optically detecting, e.g. imaging the compartments, preferably with a microscope, preferably creating imaging data thereby. Suitable microscopy techniques include bright field, fluorescence, confocal, DIC, and dark field microscopy. Other detection techniques are impedance measurements and electric conductivity measurements. Preferably, identification is carried out while the compartments are in the tube.

Standard conditions refer to the standard conditions at the respective stage.

The method may further include: analyzing the imaging data with an object recognition algorithm, preferably an artificial intelligence object recognition algorithm, to identify plant cellular objects that have transferred to the respective state.

The method may further include: dispensing a plurality of plant cellular objects that are embedded in the compartments of sample fluid, preferably in accordance with the identifiers of the respective compartments. The plant cellular objects of respective compartments may be dispensed at different target sites (e.g., into different receptacles such as different wells of a multiwell plate or into different test tubes), preferably in accordance with the identifiers of the respective compartments. The plant cellular objects may then be subjected to further treatment.

Dispensing the plurality of plant cellular objects may comprise providing:
a target surface that is wettable with the sample fluid and/or absorbent for the sample fluid, preferably wherein the surface is poorly wettable with and/or poorly absorbent for the carrier fluid; or
a target surface that is absorbent for the carrier fluid but poorly absorbent for the sample fluid; or
a target surface that is less absorbent for the sample fluid than for the carrier fluid; or
a target surface that is wettable with the sample fluid and/or absorbent for the sample fluid and wherein the surface is less wettable with and/or less absorbent for the carrier fluid.

Dispensing the plurality of plant cellular objects may include:
i. providing the microfluidic conduit with an opening through which the plant cellular objects are dispensed;
ii. flowing the sequence of compartments through the microfluidic conduit towards the opening and dispensing the compartment closest to the opening in the direction of flow through the opening at a plurality of target sites, preferably wherein the opening is positioned such that drops emerging from the opening contact the respective target site when still in contact with a wall of the microfluidic conduit and/or with the fluid in the microfluidic conduit.

Dispensing the plurality of plant cellular objects may further include:
iii. moving the opening relative to the first target site to position the opening at a second target site;
iv. repeating steps ii and iii in order to deposit plant cellular objects embedded in different compartments of sample fluid at different target sites.

The method described in the two paragraphs above may include that the microfluidic conduit comprises an inlet and an outlet, wherein the opening is located between the inlet and the outlet, the method further comprising the following steps:
providing fluid via the inlet at an input volumetric flow rate Qᵢₙ and simultaneously removing fluid via the outlet at an output volumetric flow rate Qₒᵤₜ;
wherein the input volumetric flow rate Qᵢₙ is larger than the output volumetric flow rate Qₒᵤₜ, the difference resulting in a discharge volumetric flow rate Q_{deposit} of sample fluid through the opening that results in deposition of the compartments of sample fluid embedded in the carrier fluid at the target site.

Suitable devices for dispensing the cellular objects in this manner are disclosed in co-pending European patent application no. 21 168 659.7, which is incorporated herein by reference in its entirety.

The following aspects are preferred embodiments of the description:
1. Method for developing and/or reprogramming plant cellular objects comprising the steps:
   a) providing a reservoir containing a medium with plant cellular objects;
   b) providing a first set of compartments of sample fluid embedded in carrier fluid in a microfluidic conduit, wherein the carrier fluid is immiscible with the medium, wherein the first set's compartments of sample fluid each comprise medium and at least one plant cellular object;
   c) providing one or more first state triggers to the plant cellular objects in the microfluidic conduit for inducing a first state in the plant cellular objects of the first set of compartments;
   d) incubating the plant cellular objects of the first set of compartments in the microfluidic conduit for a time span sufficient for the plant cellular objects to transfer to the first state;
   e) selecting one or more first selection parameters indicative of the first state;
   f) identifying, within the first set of compartments in the microfluidic conduit, compartments according to the one or more first selection parameters and optionally assigning the compartments with respective state identifiers.
2. The method of the preceding aspect, further comprising the step:
   g) sorting the first set's compartments according to their state as identified in step f), e.g. separating the compartments identified in step f) as comprising plant cellular objects that have, according to the one or more first selection parameters, transferred to the first state from the compartments that were not identified in step f) as comprising plant cellular objects that have transferred to the first state, preferably sorting the first set's compartments according to their state identifiers.
3. The method of any one of the preceding aspects, further comprising:
   h) providing a second set of compartments of sample fluid embedded in carrier fluid in the microfluidic conduit, wherein the second set's compartments of sample fluid each comprise medium and at least one plant cellular object;
   i) providing one or more second state triggers to the second set's plant cellular objects in the microfluidic conduit for inducing a second state in the plant cellular objects of the second set of compartments;
   j) incubating the plant cellular objects of the second set of compartments in the microfluidic conduit for a time span sufficient for the plant cellular objects to transfer to the second state;
   k) selecting one or more second selection parameters indicative of the second state;
   l) identifying, within the second set of compartments in the microfluidic conduit, compartments with plant cellular objects that have transferred to the second state and optionally assigning the compartments with respective state identifiers.
4. The method of the preceding aspect, further comprising the step:
   m) sorting the second set's compartments according to their state as identified in step l), e.g. separating the compartments identified in step I) as comprising plant cellular objects that have, according to the one or more second selection parameters, transferred to the second state from the compartments that were not identified in step l) as comprising plant cellular objects that have transferred to the second state, preferably sorting the second set's compartments according to their state identifiers.
5. Method for developing and/or reprogramming, plant cells comprising the steps:
   a) providing a reservoir containing a medium with plant cellular objects;
   b) providing several sets of compartments of sample fluid embedded in carrier fluid in a microfluidic conduit, wherein the carrier fluid is immiscible with the medium, wherein the compartments of sample fluid comprise at least one plant cellular object each;
   c) providing respective state triggers to the plant cellular objects in the microfluidic conduit for inducing respective states in the plant cellular objects;
   d) incubating the plant cellular objects in the microfluidic conduit for a time span sufficient for the plant cellular objects to transfer to the respective state;
   e) for each state, selecting one or more selection parameters indicative of the state;
   f) identifying, within the several sets of compartments, compartments with plant cellular objects that have transferred to the respective state(s) and optionally assigning the compartments with a state identifier.
6. The method of the preceding aspect, further comprising the step:
   g) sorting the compartments according to their states as identified in step f), e.g. separating the compartments identified in step f) as comprising plant cellular objects that have transferred to the respective state from the compartments that were not identified in step f) as comprising plant cellular objects that have transferred to the respective state, preferably sorting the compartments according to their state identifiers.
7. The method of any one of the preceding aspects, wherein the plant cellular objects are selected from: protoplasts, microspores, fragments originating from whole plants, such as fragments from root, stem, petals, leaves, etc., suspension or callus cells small multicellular structures such as embryos and organoids, and plant pollen cells.
8. The method of any one of the preceding aspects, comprising:
   assigning a compartment identifier and/or a set identifier to the compartments.
9. The method of any one of the preceding aspects, further comprising the step:
   transferring the sample fluid compartments to a subsequent procedural step in accordance with:
      i. their compartment identifier;
      ii. their set identifier; and/or
      iii. their state identifier;
   and/or
   discarding the compartments with a state identifier that indicates that the compartment was not identified as comprising plant cellular objects that have transferred to the respective state.
10. The method of any one of the preceding aspects, wherein the sample fluid compartments and/or sets of sample fluid compartments are treated individually, preferably according to their compartment identifier and/or set identifier and/or state identifier.
11. The method of any one of the preceding aspects, wherein the trigger(s) include(s):
   changing the composition of the sample fluid in the compartments as compared to the medium upon formation of the sample fluid compartments; and/or
   changing the composition of the sample fluid in the compartments after formation of the sample fluid compartments while the compartments are in the microfluidic channel.
12. The method of the preceding aspect, wherein changing the composition includes adding a manipulation fluid comprising a trigger substance.
13. The method of the preceding aspect, wherein different sets of sample compartments are provided with different trigger substances.
14. The method of any of the two preceding aspects, wherein different compartments of sample fluid within the first and/or second set are provided with the same trigger substance at different concentrations.
15. The method of any one of the preceding aspects, wherein the compartments comprise 1000 or less, 200 or less, preferably 50 or less, more preferably 10 or less plant cellular objects per compartment.
16. The method of any one of the preceding aspects, wherein the compartments each have a volume of:
   at least 10-30 nL, or 30-60, or at least 60-100 nL;
   at least, 10 nL, at least 30 nL, at least 60 nL, or at least 100 nL; and/or
   10 ul or less, preferably 5 μl or less.
17. The method of any one of the preceding aspects, wherein the compartments each have a number density of 50 plant cellular objects per 100 nL compartment volume.
18. The method of any one of the preceding aspects, wherein the state trigger comprises at least one substance selected from the group comprising culture medium salts or organic components, small chemical molecules, plant growth regulators or macromolecules, temperature and light regimes, or combinations of different triggers.
19. The method of any one of the preceding aspects, wherein the state trigger comprises Trichostatin A, optionally at a concentration of 0.1 μM to 0.35 μM in the compartments.
20. The method of any one of the preceding aspects, wherein the step(s) of providing the state trigger(s), i.e. step(s) c)/i), include(s) subjecting at least some of the compartments and/or at least one of the sets of compartments to light shock, heat shock and/or cold shock conditions for inducing the respective state, preferably wherein:
   the heat shock conditions include an elevated temperature of at least 5° C. above standard incubation temperature, for example at least 32° C., preferably wherein the elevated temperature is maintained for at least 2 days;
   the cold shock conditions include a lowered temperature of at least 5° C. or 10° C. below standard incubation temperature, preferably wherein the lowered temperature is maintained for at least 2 days, and/or
   the light shock includes a change of the lighting conditions as compared to standard incubation.
21. The method of any one of the preceding aspects, wherein the microfluidic conduit is a tube, wherein the tube is preferably at least 10 cm long, more preferably at least 50 cm long, more preferably at least 100 cm long, most preferably at least 500 cm long.
22. The method of the preceding aspect, wherein the tube material is at least partially transparent, and/or has a gas permeability for CO₂ that is sufficient for supplying the plant cellular objects with the necessary amount of CO₂, and/or low wetting with water.
23. The method of any one of the two preceding aspects, wherein the tube material is polymeric, preferably wherein the tube material comprises or consists of fluorethylen-propylen or polytetrafluorethylene (PTFE).
24. The method of any one of the preceding aspects, wherein the plant cellular objects are haploid microspore stem cells, for example haploid microspore stem cells of Brassica napus; and/or wherein the potentially induced state is a plant embryo.
25. The method of any one of the preceding aspects, wherein the step(s) of providing one or more sets of compartments, i.e. step(s) b)/h), include(s): providing at least 100, preferably at least 1000, more preferably at least 10000 compartments per microfluidic conduit.
26. The method of any one of the preceding aspects, wherein each set comprises at least 1, at least 2, at least 5, at least 10, at least 100, at least 1000 or at least 10000 compartments of sample fluid.
27. The method of any one of the preceding aspects, wherein the step(s) of providing one or more sets of compartments, i.e. step(s) b)/h), include(s):
   ix. providing a sample of dispersed plant cellular objects in the medium;
   x. providing the microfluidic conduit with an inlet, an outlet and an opening located between the inlet and the outlet;
   xi. providing the carrier fluid via the inlet to the microfluidic conduit at an input volumetric flow rate (Qᵢₙ) and removing fluid from the microfluidic conduit via the outlet at an output volumetric flow rate (Qₒᵤₜ);
   xii. positioning the opening in the medium;
   xiii. setting the input volumetric flow rate (Qᵢₙ) to be smaller than the output volumetric flow rate (Qₒᵤₜ), the difference resulting in a takeup volumetric flow rate (Qₜₐₖₑᵤₚ) of medium through the opening that results in the medium being embedded in the carrier fluid as the sample fluid compartments.
28. The method of the preceding aspect, wherein the step(s) of providing one or more sets of compartments, i.e. step(s) b)/h), further include(s):
   xiv. selecting a target plant cellular object among the plant cellular objects and changing a position of the opening relative to the target plant cellular object from a starting relative position to a target position to bring the opening proximate to the target plant cellular object;
   xv. drawing the target plant cellular object into the microfluidic conduit together with a respective compartment of sample fluid when embedding the compartment of sample fluid in the carrier fluid according to step v);
   xvi. repeating steps i)-vii) until a desired number of compartments has been created.
29. The method of the preceding aspect, wherein the microfluidic conduit comprises a manipulation inlet arranged at the opening or between the opening and the outlet, and wherein the method further comprises: delivering a manipulation fluid via the manipulation inlet into a respective compartment in the microfluidic conduit at a manipulation volumetric flow rate Qₘₐₙᵢₚ, wherein the manipulation fluid optionally comprises a trigger substance.
30. The method of any of aspects 1-26, wherein the step(s) of providing one or more sets of compartments, i.e. step(s) b)/h), include(s):
   i) providing a microfluidic manifold comprising a microchannel, the microchannel being in fluid communication with the microfluidic conduit, a carrier fluid supply, the reservoir, and optionally a further fluid supply;
   ii) adjusting a flow rate from the carrier fluid supply to the manifold, a flow rate from the reservoir to the manifold, and optionally a flow rate from the further fluid supply to the manifold such that compartments of sample fluid embedded in carrier fluid are created in the microchannel of the manifold, wherein the compartments of sample fluid each comprise medium and at least one plant cellular object;
   iii) transferring the compartments of sample fluid from the microchannel of the manifold to the microfluidic conduit.
31. The method of any one of the preceding aspects, wherein incubation step d) includes:
   maintaining the temperature of the compartments above standard incubation temperature, preferably for at least 2 hours, at least 5 hours, at least 8 hours, at least 12 hours, at least 24 hours, at least 2 days, at least 3 days, at least 5 days or at least 6 days; and/or
   reverse light conditions as compared to standard light conditions;
   one or more further conditions, e.g. CO₂ concentration, which deviate from respective standard conditions.
32. The method of any one of the preceding aspects, wherein step e) includes:
   Optically detecting, e.g. imaging the compartments, preferably with a microscope, while the compartments are in the tube, preferably creating imaging data thereby.
33. The method of the preceding aspect, further including:
   analyzing the imaging data with an object recognition algorithm, preferably an artificial intelligence object recognition algorithm, to identify plant cellular objects that have transferred to the respective state.
34. The method of any one of the preceding aspects comprising:
   dispensing a plurality of plant cellular objects that are embedded in the compartments of sample fluid, preferably in accordance with the identifiers of the respective compartments.
35. The method of the preceding aspect, wherein the plant cellular objects of respective compartments are dispensed at different target sites (e.g., into different receptacles such as different wells of a multiwell plate or into different test tubes), preferably in accordance with the identifiers of the respective compartments.
36. The method of any of the two preceding aspects, wherein dispensing the plurality of plant cellular objects comprises providing:
   a target surface that is wettable with the sample fluid and/or absorbent for the sample fluid, preferably wherein the surface is poorly wettable with and/or poorly absorbent for the carrier fluid; or
   a target surface that is absorbent for the carrier fluid but poorly absorbent for the sample fluid; or
   a target surface that is less absorbent for the sample fluid than for the carrier fluid; or
   a target surface that is wettable with the sample fluid and/or absorbent for the sample fluid and wherein the surface is less wettable with and/or less absorbent for the carrier fluid.
37. The method of any of the three preceding aspects, wherein dispensing the plurality of plant cellular objects comprises:
   i. providing the microfluidic conduit with an opening through which the plant cellular objects are dispensed;
   ii. flowing the sequence of compartments through the microfluidic conduit towards the opening and dispensing the compartment closest to the opening in the direction of flow through the opening at a plurality of target sites, preferably wherein the opening is positioned such that drops emerging from the opening contact the respective target site when still in contact with a wall of the microfluidic conduit and/or with the fluid in the microfluidic conduit.
38. The method of the preceding aspect, wherein dispensing the plurality of plant cellular objects further comprises:
   iii. moving the opening relative to the first target site to position the opening at a second target site;
   iv. repeating steps ii and iii in order to deposit plant cellular objects embedded in different compartments of sample fluid at different target sites.
39. The method of any of the two preceding aspects, wherein the microfluidic conduit comprises an inlet and an outlet, wherein the opening is located between the inlet and the outlet, the method further comprising the following steps:
   providing fluid via the inlet at an input volumetric flow rate Qᵢₙ and simultaneously removing fluid via the outlet at an output volumetric flow rate Qₒᵤₜ;
   wherein the input volumetric flow rate Qᵢₙ is larger than the output volumetric flow rate Qₒᵤₜ, the difference resulting in a discharge volumetric flow rate Q_{deposit} of sample fluid through the opening that results in deposition of the compartments of sample fluid embedded in the carrier fluid at the target site.
40. The method of any of the preceding aspects, wherein the carrier fluid is immiscible with the sample fluid(s).

The invention is further explained with reference to the following protocols and examples and with reference to the figures, in which

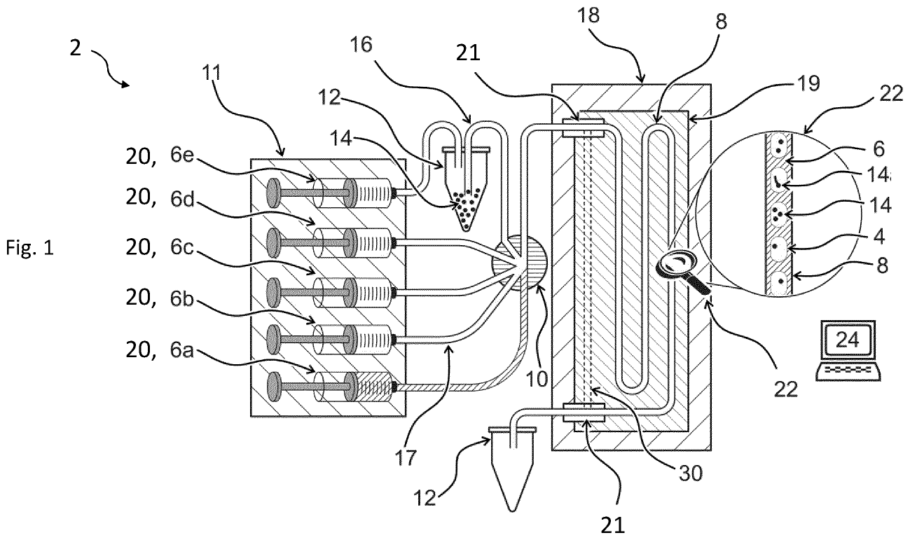

FIG. 1 shows an experimental setup scheme for compartment sequence generation;

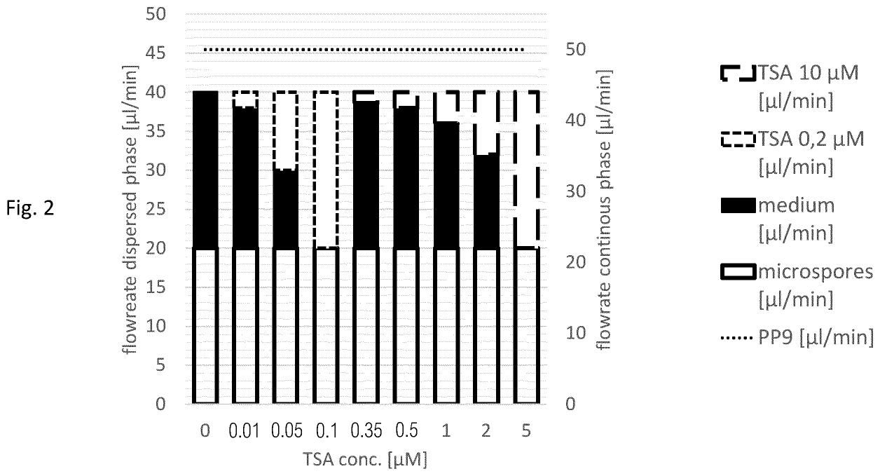

FIG. 2 shows a program for generation of nine concentration combinations of TSA to a compartment sequence;

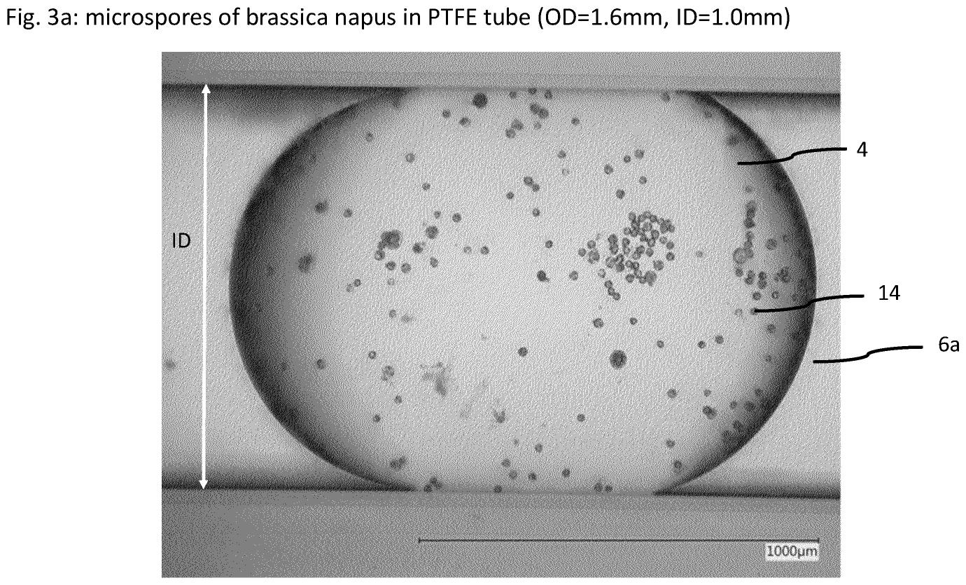

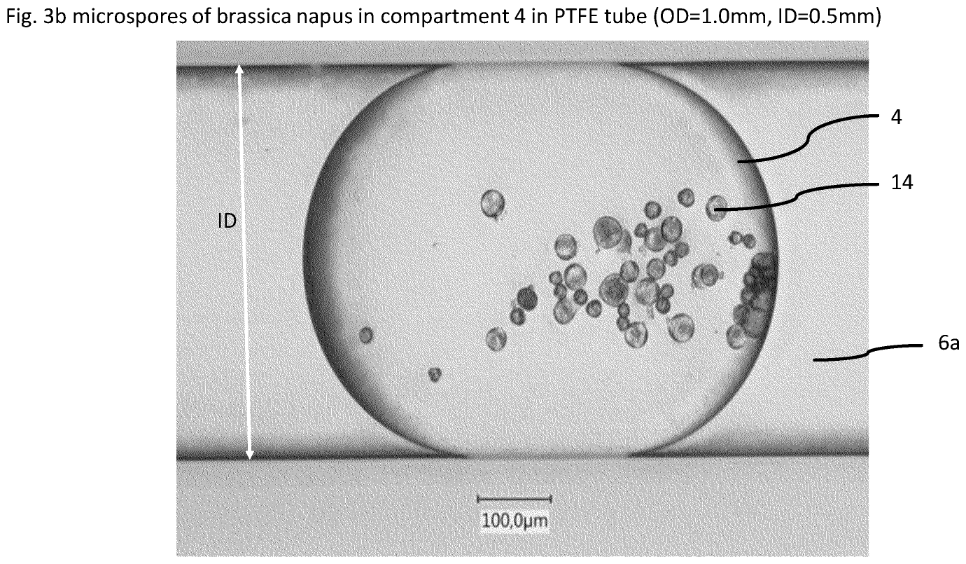

FIG. 3a shows a microscopy image of a compartment with microspores of Brassica napus in a tube;

FIG. 3b shows a microscopy image of another compartment with microspores of Brassica napus in another tube;

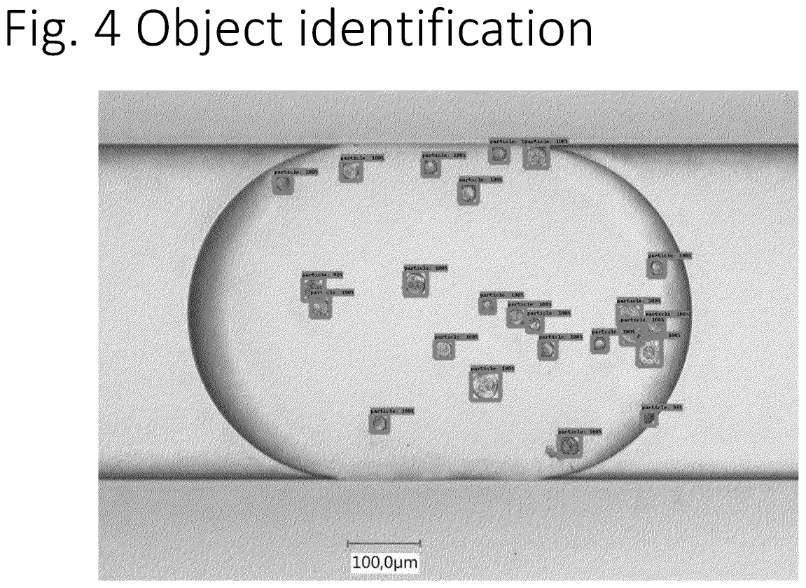

FIG. 4 shows a microscopy image of compartment with microspores identified by an algorithm;

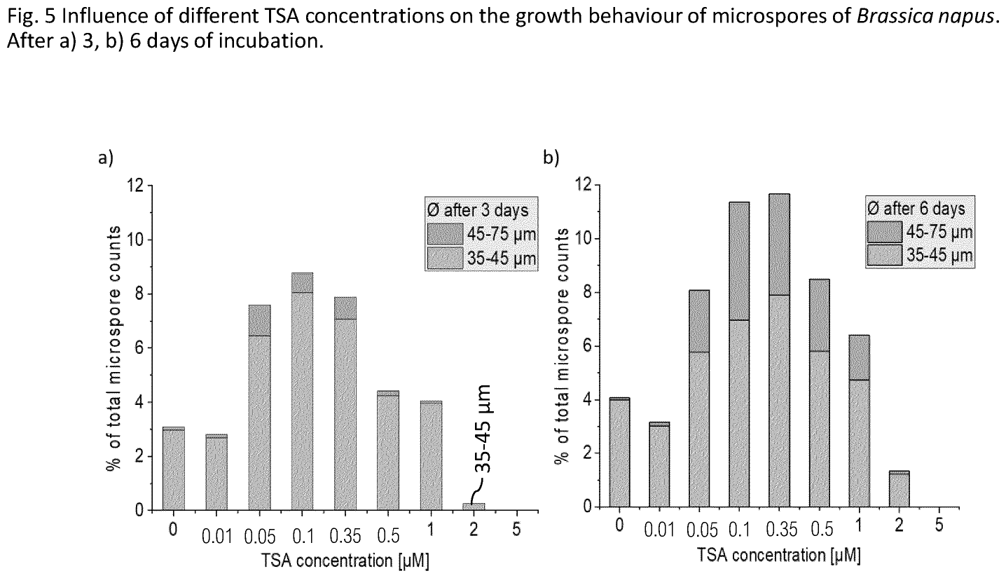

FIG. 5 shows results of an example;

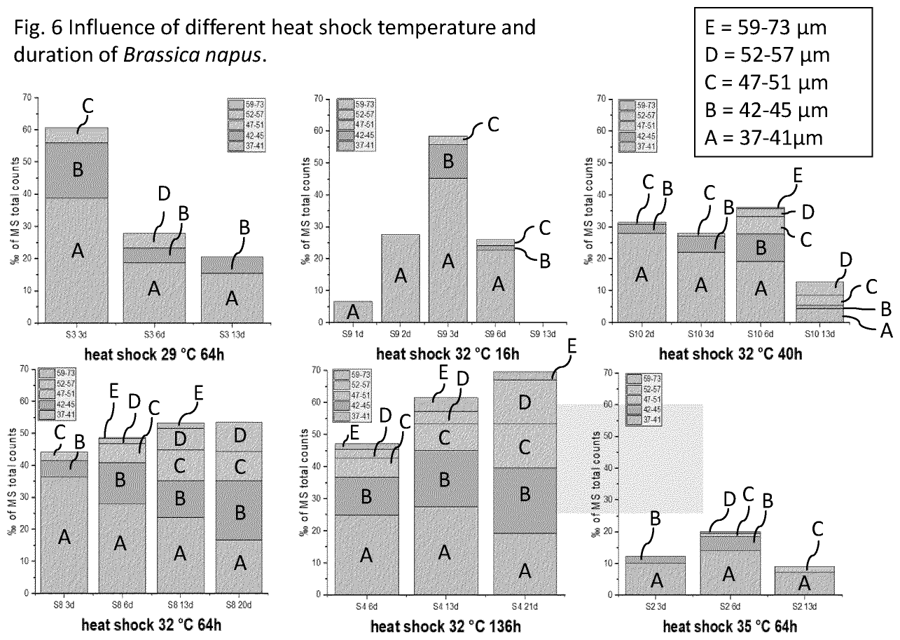

FIG. 6 shows results of an example.

FIG. 1 shows a scheme of a setup 2 suitable for the present invention, i.e. for providing, particularly generation of, compartments 4 embedded in carrier fluid 6 in a microfluidic conduit 8. A compartment generator 10 may be connected to a feeding device 11, e.g. a pump. As shown, the compartment generator 10 may comprise five inlet channels and one outlet channel, and the feeding device 11 may be a 5-channel precision syringe pump 11. Other numbers of channels and/or types of devices are contemplated.

Compartment generators are also generally known as droplet generators. Herein, the term “droplet” refers to a volume of a first phase surrounded by a second phase. Additional phases, e.g. a single layer of lipids or a double layer of lipids, are contemplated but not necessary. “Droplet” is used as a synonym for “compartment”.

A container 12 may be used to carry plant cellular objects 14, e.g. cells 14, e.g. microspores or protoplasts or other cells or cell clusters derived e.g. from dicotyledonous plants like Brassica napus or others, or monocotyledonous plants like maize, or mosses or others. The container 12 may be a vial 12. The plant cellular objects 14 may settle down in the tip of the vial 12 and may be fed to the compartment generator 10 through an immersed container outlet conduit 16, here a tube 16. This may be effected by feeding a fluid 6e, e.g. medium or carrier fluid 6, into the fluid-tightly sealed container 12, e.g. from one of the channels of the feeding device 11. Fluids 6a-6d from the other channels of the feeding device 11 may be fed from the feeding device's channels to the compartment generator 10 via respective conduits 17. The generated compartment 4 sequence, or set(s) of compartments 4, may be collected in the microfluidic conduit 8, which may be a tube and/or be comprised in an observation unit 18. The microfluidic conduit 8 may be connected to another vial 12 configured for collecting waste exiting the microfluidic conduit 8. The observation unit 18 may comprise the microfluidic conduit/tube 8 inside a transparent frame 19 configured for observation under an optical microscope 22. However, other configurations are contemplated.

The microfluidic conduit/tube 8 may be disconnected from the compartment generator 10. The microfluidic conduit/tube 8 may be closed at both of its ends, e.g. for observation, i.e. identification steps, and/or incubation. For example, the system may comprise a connector conduit 30 bypassing the compartment generator 10 and/or the waste collecting vial 12, and connecting the microfluidic conduit 8 to form a closed loop. Switching between a connection to the compartment generator 10 and a connection to the connector conduit 30 may occur via a fluid connector 21. Switching between a connection to the waste vial 12 and a connection to the connector conduit 30 may occur via a fluid connector 21. Fluid connectors 21, which are configured for switching between channels, are generally known in the art. The observation unit 18 may be configured for positioning, e.g. under a microscope 22, particularly for automated positioning, e.g. by a robotic arm. The observation unit 18 may be configured for incubation of plant cellular objects 14 in the microfluidic conduit 8 in the observation unit 18.

The compartments 4 may be optically detected, particularly imaged with a microscope 22. The microscope 22 is indicated by the magnifying glass symbol in FIG. 1. The inset schematically shows a detail, i.e. a magnified portion, of the microfluidic conduit 8 including some compartments 4 embedded in carrier fluid 6, the compartments comprising plant cellular objects 14. Per compartment 4 a series of Z-Stack images with different z-axis focus points may be taken and used for analysis, i.e. for the identification step(s). The z-axis is defined as the optical axis of the microscope 22. The images may be analyzed, e.g. with image analysis software of a computer 24 or the like.

The concentration of the plant cellular objects 14 in the compartments 4 may be regulated as adequate upon formation. For example, the flow rate of the medium including the plant cellular objects during formation of the compartments 4 may be regulated to provide a desired portion of the compartments 4, and thus a desired concentration of plant cellular objects 14 in the compartments 4. The same applies, if used, for trigger(s) and/or other further fluids to be added to the compartments 4. If the sum of fluids does not amount to the desired compartment volume, the difference may be compensated by providing the option of adding medium without plant cellular objects 14 to the compartments 4 and regulating the flow rate of medium accordingly. Additionally, or alternatively, a second container 12 with plant cellular objects 14 in medium at a different concentration may be integrated into the system 2, analogous to the first container 12 described above. Depending on the desired concentration, solution from the first and/or the second container 12 at an adequate flow rate may be fed into the compartment generator 10, analogous to the trigger solutions.

### EXAMPLES

In the examples shown here the cultivation of microspores of Brassica napus as plant cellular objects is shown. Recipes of materials are provided at the end of the example.

### Example 1

### 1. Microfluidic Procedure: Microspore Encapsulation, Compartment Generation and Cultivation in Tube-Based Microfluidic System

For the cultivation of the microspores, the desired compartment sequences were generated by application of the system as shown in FIG. 1. The core of the system 2 was a five-channel precision syringe pump 11 (NEMESYS Cetoni GmbH, Germany) which can feed up to five fluids 6a-6e carried in glass syringes 20 (SETonic GmbH, Germany). One channel was used for the delivery of perfluoro-methyldecalin (PP9) as carrier fluid 6/6a, which is immiscible with the other fluids and thus separates the individual aqueous compartments 4 from one another inside the generated compartment 4 sequence. Another channel was used for 1×NLN13 (recipe below) medium 6b to dilute the medium with the microspores 14 and adjust the total volume of the compartments 4, and thus the concentration of the plant cellular objects 14 and the other substances, during formation. Two different trigger solutions 6c, 6d were used in channels three and four. Channel five was used for dosing the cell suspension, i.e. the medium with the plant cellular objects 14, itself. A cell container vial 12 was employed to feed the microspores 14 into the compartment generator 10. For this, a small conical glass vial 12 with a capacity of about 2 ml was used (See FIG. 1). The vial 12 was filled with the microspore suspension, i.e. medium with plant cellular objects 14, and closed airtight and free of air bubbles. The initial microspore density in the vial 12 was about 700.000 microspores/ml.

By syringe pump-controlled addition of medium 6e into the container 12 the suspended microspores 14 were fed through the container outlet conduit 16, a 0.2 mm ID FEP tube, into the compartment generator 10. ID stands for inner diameter. Instead of medium 6e it is also contemplated to add carrier fluid 6 to the container 12 with the suspended microspores. The individual syringes 20 were connected with the compartment generator 10 via respective conduits 17. Compartments 4 of about 120 nL volume each were formed and forwarded in a 0.5 mm ID PTFE tube as the microfluidic conduit 8 for incubation. A detailed description of the used compartment generator 10 can be found in “Cao J, Richter F, Kastl M, Erdmann J, Burgold C, Dittrich D, Schneider S, Köhler J M, Groß GA (2020) Droplet-Based Screening for the Investigation of Microbial Nonlinear Dose-Response Characteristics System, Background and Examples. Micromachines. 10.3390/mi1106057”, which is incorporated herein in its entirety by reference. For sufficient diffusive aeration during long-time incubation times, PTFE tubes with low wall thickness were used (inner diameter: 0.5 mm, outer diameter: 1.0 mm). Typical flow rate conditions of about 30 μl/min for the aqueous phases, i.e. the sample fluid, i.e. the medium 6b, the medium with the plant cellular objects, and the trigger solutions 6c, 6d, and about 100 μl/min for the embedding phase, i.e. the carrier fluid 6a, were used. All other connecting tubes, i.e. the conduits 17 and the container outlet conduit 16, were made of FEP (fluorinated ethylene propylene) and had an outer diameter OD of 1.6 mm and an inner diameter ID of 0.5 mm. However, other tube materials and dimensions may be used.

For a dose-response experiment the Trichostatin A (TSA) was chosen as a trigger. The stimulating effect of TSA on the germination of the microspores 14 was investigated in a concentration range from 0.01 μM up to 5 μM. Therefore, TSA stock solutions of 0.2 μM TSA 6c and 10 μM TSA 6d were used in the syringes 20 for triggers 1 and 2. The application of an appropriate flow rate program yielded the compartment 4 sequence with the desired concentrations of TSA in the compartments 4, as shown in FIG. 2. The resulting compartments 4 were composed of 50% volume microspore suspension, 50%-0% dilution medium 6b and 0-50% of the trigger solution 6c, 6d. Depending on the intended concentration of TSA in the generated compartments 4, either 0.2 μM TSA solution 6c or 10 μM TSA solution 6d was added to the generated compartments 4. The flow rate for the carrier medium 6e, here PP9, was kept constant at 50 μl/min. In FIG. 2, the white part of the columns with continuous black contour symbolizes the flow rate of the microspore suspension, which was kept at a constant flow rate of 20 μl/min. The white portions with dashed black contours represent the two TSA solutions 6c, 6d and vary within a flow rate range of 0-20 μl/min to adjust the concentration. The medium 6b, shown in black in FIG. 2, is used to compensate for the difference to 20 μl/min. This results in a constant total flow rate of the dispersed phase of 40 μl/min. “Dispersed phase” refers to the combined aqueous phases, i.e. trigger solution, microspores in medium, and additionally added medium, and constitutes the sample fluid at formation of the compartments 4. For each value of TSA concentration, a set of compartments 4 with about 150 compartments 4 was generated. For all nine investigated TSA concentrations values about 1.500 compartments were generated in the compartment generator 10 within 5 min in total. The nine investigated compartment compositions are also shown in Table 1.

TABLE 1

| TSA conc. | microspores | medium | TSA 0.2 μM | TSA 10 μM | PP9 |
|---|---|---|---|---|---|
| [μM] | [μl/min] | [μl/min] | [μl/min] | [μl/min] | [μl/min] |
| 0 | 20 | 20 | 0 | 0 | 50 |
| 0.01 | 20 | 18 | 2 | 0 | 50 |
| 0.05 | 20 | 10 | 10 | 0 | 50 |
| 0.1 | 20 | 0 | 20 | 0 | 50 |
| 0.35 | 20 | 18.6 | 0 | 1.4 | 50 |
| 0.5 | 20 | 18 | 0 | 2 | 50 |
| 1 | 20 | 16 | 0 | 4 | 50 |
| 2 | 20 | 12 | 0 | 8 | 50 |
| 5 | 20 | 0 | 0 | 20 | 50 |

As already mentioned, the compartments 4 were fed from the compartment generator 10 into the microfluidic conduit 8 for incubation and identification. Incubation and identification took place in observation unit 18. For optical identification, a microscope 22 was used.

In addition to the TSA trigger, a heat shock trigger was applied. The compartments 4 were subjected to 32° C. for 64 h. The timely sequence of triggers was as follows: Generation of compartments 4 including microspores 14 and TSA trigger at 0 h, heat shock at 0 h-64 h, and (further) incubation at standard incubation temperature.

Identification of microspores 14 that had transferred to another state was implemented by imaging the compartments with a digital microscope in bright field transmission at 300× magnification and analyzing the images with a software. Particularly, one Z-stack per droplet were taken and saved for further processing.

FIG. 3a shows a microscopy image of a compartment 4 with microspores 14 of Brassica napus in a PTFE tube with an inner diameter ID of 1.0 mm and an outer diameter OD of 1.6 mm. FIG. 3b shows a microscopy image of another compartment 4 with microspores 14 of Brassica napus in a PTFE tube with an inner diameter ID of 0.5 mm and an outer diameter OD of 1.0 mm, showing that the invention may be implemented with different tube diameters.

Images were taken at formation of the compartments 4, i.e. at the time points of interest for each concentration of TSA (see results of Example 1 below).

### Data Evaluation

Image processing was done with an artificial intelligence-based particle detection program. However, any suitable image processing may be used. The program analyzed the images and identified plant cellular objects 14 in the compartments 4 as well as their sizes in terms of their diameters.

The artificial intelligence-based particle detection program was able to measure the microspore sizes and display them as an overlay to the analyzed image, as shown in FIG. 4, and as a size distribution. The values of the size distribution are presented as stacked bar charts, e.g. like in FIG. 5, which shows the results of Example 1 below. The diagrams each show a section of the total size distribution of the microspores 14 after 3 days (a)) and after 6 days (b)). The columns are again subdivided according to size. The lower part in lighter grey summarizes all microspores with diameters from 35 μm to 45 μm and the upper part in darker grey represents microspores 14 between 45 and 75 μm and of 75 μm. The 35 μm was chosen as the lower limit, as the microspores 14 had a size of about 15-30 μm at the beginning of the culture (before the heat shock), so with a certain tolerance one can assume cultivation-related growth by cell division from 35 μm. The increase in size can be used with caution as an indication of reprogramming as well as the beginning of embryogenesis and thus as a selection parameter.

With the initial division and one or two subsequent divisions, the microspores can reach sizes of about 40-45 μm, as was demonstrated in Solis M-T, El-Tantawy A-A, Cano V, Risueño MC, Testillano PS (2015) “5-azacytidine promotes microspore embryogenesis initiation by decreasing global DNA methylation, but prevents subsequent embryo development in rapeseed and barley”; Frontiers in plant science, 472. 10.3389/fpls.2015.00472, which is incorporated herein by reference in its entirety. Therefore, 45 μm was chosen as the threshold for grouping the measured microspore sizes into two groups. The upper limit, 75 μm, represents the maximum size measured in all experiments after six days of cultivation.

### 3. Results of Example 1

The results of the image analysis are shown in FIG. 5.

From 0.01 μM to 5 μM TSA, the bar charts show a clear, constant course similar to a bell curve. After 3 days incubation at 32° C., about 3% microspores larger than 35 μm could be measured at 0 and 0.01 μM TSA. The percentage first rises steadily with increasing TSA concentration, with approximately 7.6% at 0.05 M and 9% at 0.1 μM. From 0.35 μM TSA onwards the percentage decreases again, first to 8%, then to about 4.4% and 4% at 0.5 μM and 1 μM, respectively, and finally to almost 0% at 2 μM and 0% at 5 μM. A closer look at the individual columns shows the main proportion of microspores in a size range between 35 and 45 μm. Only at 0.05, 0.1 and 0.35 μM TSA, about 1% of the total number of microspores is larger than 45 μm and thus already in a multicellular stage. After 6 days, the percentages for 0.01 and also for the reference 0 μM TSA have increased only marginally and especially the percentage of microspores larger than 45 μm has remained almost constant. This suggests that following a possible initiation of embryogenesis after the heat shock no further growth took place. In contrast, a clear growth can be seen between the concentration levels 0.05-1 μM. At 0.05 μM TSA the total column shows hardly any increase, but the ratio of the proportions shifts towards the size range above 45 μm. This suggests further growth of already reprogrammed microspores, new microspores that only started to divide and grow at a later time are hardly visible. The result at 0.5 μM is very similar to that just described but shows almost a doubling of the total column height and a significant increase in the proportion above 45 μm diameter. For both concentrations, 0.05 μM and 0.5 μM TSA, the percentage of total microspores with a diameter of at least 35 μm is about 8% with about 2% of very large microspores (>45 μm). At 0.5 μM as well as at the concentrations 0.1 and 0.35 μM, the most significant changes can be seen, both in the total number and in the percentage ratios. At 0.1 μM the total column increases by approximately 2% to 11.4% and the proportion of microspores larger than 45 μm increases by about 3% to 4.4%. At 0.35 μM, an increase from 8% to 11.7% can be seen for microspores larger than 35 μm, with 3.8% of the microspores being very large. At 1 μM the total column height increased to 6.4% and the green portion to approximately 1.7%. For 2 μM a slight growth to about 1.3% microspores larger than 35 μm can be observed, 5 μM, without any recognizable growth, emerged as the lethal dose.

In general, the results show the strongest growth between 0.1 and 0.35 μM TSA as a result of the screening method. Lower concentrations have a weaker effect, with higher concentrations TSA has an increasingly harmful effect.

### Example 2

Compartments 4 with microspores of Brassica napus were created as in Example 1, with the following difference: Instead of providing a trigger solution with TSA to the compartments, the trigger was chosen to be a heat shock only.

Three temperatures, 29° C., 32° C. and 35° C., combined with four different incubation durations, 16 h, 40 h, 64 h and 136 h, were tested as the respective heat shock condition for the present experiment as summarized in Tab. 1. It is noted that this is an example, where the method steps of providing a trigger and the incubation step are partially carried out simultaneously.

TABLE 2
Overview of tested heat shock conditions

| Heat shock temperature [° C.] | Heat shock duration [h] |
|---|---|
| 29 | 64 |
| 32 | 16 |
| | 40 |
| | 64 |
| | 136 |
| 35 | 64 |

Incubation times were selected such that analysis, i.e. imaging, of the compartments occurred at 3 days, 6 days and 13 days after start of the heat shock. For example, with 64 hours of heat shock duration, a further incubation time of additional 8h resulted in an analysis at 72 hours, i.e. 3 days, after the start of the heat shock.

Image acquisition and analysis, i.e. compartment identification, was done as described in Example 1.

Again, the size of the microspores was selected as the selection parameter. The size indicates the next state, namely first division (37-45 μm specified as column A and B) and further division (47-73 μm specified as column C, D and E).

The results are shown in FIG. 6 and in the following Table 3:

| | Temperature | | | |
|---|---|---|---|---|
| | heat-shock | Duration of | | |
| Tube | [° C.] | heat-shock | After 3 days | After 6 days |
| MTP/ | 32 | 64 h | +++ | +++ |
| reference | | | | |
| nL-Droplet | 32 | 16 h | +++ | + |
| | 32 | 40 h | ++ | ++ |
| | 29 | 64 h | +++ | + |
| | 32 | 64 h | +++ | +++ |
| | 35 | 64 h | + | + |
| | 32 | 136 h | Not | ++++ |
| | | | microscoped | |

In the table, the number of plus-symbols, +, ++, +++, ++++, indicates the degree of growth of the microspores 14 with a higher number indicating a higher degree of growth. “++++” indicates the highest degree and “+” the lowest degree of growth. In FIG. 6, the portions of the bars indicating the different sizes of the microspores 14 are stacked according to increasing microspore size, as shown in the insets.

Overall, the best and most sustained growth was observed at 32° ° C./64 h and 32° C./136 h, with a slight advantage in the latter.

### Recipes:

1×NLN13:
   NLN salts: 0,386 g/L (DUCHEFA BIOCHEMIE B.V, Netherlands);
   NLN vitamins: 1.03 g/L (DUCHEFA BIOCHEMIE B.V, Netherlands);
   Ca(NO₃)₂×4H₂O: 0.5 g/L (DUCHEFA BIOCHEMIE B.V, Netherlands);
   Sucrose: 130 g/L (Carl Roth, Germany);
PP9: perfluoromethyldecalin PP9 (F2 Chemicals, UK)

### LIST OF REFERENCE SIGNS

2 setup;
4 compartment/droplet;
6 carrier fluid;
8 microfluidic conduit;
10 compartment generator;
11 feeding device, pump;
12 container/vial;
14 plant cellular object/cell;
16 container outlet conduit/tube;
17 conduit from pump to compartment generator;
18 incubation and/or observation unit;
19 frame;
20 syringe;
21 fluid connector;
22 microscope;
24 computer;
30 connector conduit.

1. A method for developing and/or reprogramming plant cellular objects comprising the steps:
a) providing a reservoir containing a medium with plant cellular objects;
b) providing a first set of compartments of sample fluid embedded in carrier fluid in a microfluidic conduit, wherein the carrier fluid is immiscible with the medium, wherein the first set's compartments of sample fluid each comprise medium and at least one plant cellular object;
c) providing one or more first state triggers to the plant cellular objects in the microfluidic conduit for inducing a first state in the plant cellular objects of the first set of compartments;
d) incubating the plant cellular objects of the first set of compartments in the microfluidic conduit for a time span sufficient for the plant cellular objects to transfer to the first state;
e) selecting one or more first selection parameters indicative of the first state;
f) identifying, within the first set of compartments in the microfluidic conduit, compartments according to the one or more first selection parameters and optionally assigning the compartments with respective state identifiers.

2. The method of claim 1, further comprising the step:
g) sorting the first set's compartments according to their state as identified in step f), e.g. separating the compartments identified in step f) as comprising plant cellular objects that have, according to the one or more first selection parameters, transferred to the first state from the compartments that were not identified in step f) as comprising plant cellular objects that have transferred to the first state.

3. The method of claim 1, further comprising:
h) providing a second set of compartments of sample fluid embedded in carrier fluid in the microfluidic conduit, wherein the second set's compartments of sample fluid each comprise medium and at least one plant cellular object;
i) providing one or more second state triggers to the second set's plant cellular objects in the microfluidic conduit for inducing a second state in the plant cellular objects of the second set of compartments;
j) incubating the plant cellular objects of the second set of compartments in the microfluidic conduit for a time span sufficient for the plant cellular objects to transfer to the second state;
k) selecting one or more second selection parameters indicative of the second state;
l) identifying, within the second set of compartments in the microfluidic conduit, compartments with plant cellular objects that have transferred to the second state and optionally assigning the compartments with respective state identifiers.

4. The method of claim 1, comprising:
assigning a compartment identifier or a set identifier to the compartments.

5. The method of claim 1, further comprising the step:
transferring the sample fluid compartments to a subsequent procedural step in accordance with one or a combination of:
i. their compartment identifier;
ii. their set identifier;
iii. their state identifier.

6. The method of claim 1, wherein the sample fluid compartments or sets of sample fluid compartments are treated individually.

7. The method of claim 1, wherein the trigger(s) include(s):
changing the composition of the sample fluid in the compartments as compared to the medium upon formation of the sample fluid compartments; or
changing the composition of the sample fluid in the compartments after formation of the sample fluid compartments while the compartments are in the microfluidic channel.

8. The method of claim 7, wherein different sets of sample compartments are provided with different trigger substances.

9. The method of claim 7, wherein different compartments of sample fluid within the first or second set are provided with the same trigger substance at different concentrations.

10. The method of claim 1, wherein the state trigger comprises at least one substance selected from the group comprising culture medium salts or organic components, small chemical molecules, plant growth regulators or macromolecules, temperature and light regimes, or combinations of different triggers.

11. The method of claim 1, wherein the step of providing the state trigger(s), i.e. step(s) c)/i), include(s) subjecting at least some of the compartments or at least one of the sets of compartments to one or a combination of light shock, heat shock and cold shock conditions for inducing the respective state, wherein:
the heat shock conditions include an elevated temperature of at least 5° C. above standard incubation temperature;
the cold shock conditions include a lowered temperature of at least 5° C. or 10° ° C. below standard incubation temperature; and
the light shock includes a change of the lighting conditions as compared to standard incubation.

12. The method of claim 1, wherein the microfluidic conduit is a tube that is at least 50 cm long.

13. The method of claim 1, wherein step e) includes:
optically detecting the compartments while the compartments are in the tube, and creating imaging data thereby.

14. The method of claim 1 comprising:
dispensing a plurality of plant cellular objects that are embedded in the compartments of sample fluid in accordance with the identifiers of the respective compartments.

15. The method of claim 1, wherein the carrier fluid is immiscible with the sample fluid(s).

16. The method of claim 2, wherein step g) comprises:
sorting the first set's compartments according to their state identifiers.

17. The method of claim 3, further comprising the step:
m) sorting the second set's compartments according to their state as identified in step l).

18. The method of claim 17, wherein step m) includes separating the compartments identified in step l) as comprising plant cellular objects that have, according to the one or more second selection parameters, transferred to the second state from the compartments that were not identified in step l) as comprising plant cellular objects that have transferred to the second state.

19. The method of claim 17, wherein step m) includes sorting the second set's compartments according to their state identifiers.

20. The method of claim 1, further comprising the step:
discarding the compartments with a state identifier that indicates that the compartment was not identified as comprising plant cellular objects that have transferred to the respective state.

21. The method of claim 6, wherein the sample fluid compartments or sets of sample fluid compartments are treated according to their compartment identifier or set identifier or state identifier.

22. The method of claim 14, wherein the plant cellular objects of respective compartments are dispensed at different target sites in accordance with the identifiers of the respective compartments.
